(19) United States
(12) Patent Application Publication
(10) Pub. No.: US 2026/0315202 A1
(43) Pub. Date: Oct. 15, 2026

(54) PERSONALIZED TOOTH BRUSHING GUIDE METHOD, AND ELECTRONIC DEVICE AND STORAGE MEDIUM SUPPORTING SAME

(71) Applicant: Samsung Electronics Co., Ltd., Suwon-si (KR)

(72) Inventors: Seungyong JEONG, Suwon-si (KR); Minkyung HWANG, Suwon-si (KR)

(21) Appl. No.: 19/684,652

(22) Filed: May 21, 2026

Related U.S. Application Data
(63) Continuation of application No. PCT/KR2024/018496, filed on Nov. 21, 2024.

(30) Foreign Application Priority Data
Nov. 23, 2023 (KR) 10-2023-0164469
Jan. 4, 2024 (KR) 10-2024-0001530

(51) Int. Cl. A61C 19/04; G09B 19/00

(52) U.S. Cl. CPC A61C 19/04; G09B 19/0084

(57) ABSTRACT

An electronic device includes a wireless communication circuit, a display, a processor, and a memory for storing instructions. The instructions may be executed by the processor to cause the electronic device to: identify first data related to an electronic medical record of a user based on detection of a first external electronic device and a first object related to a tooth brushing activity; determine, based on the first data, a first tooth brushing pattern related to the tooth brushing activity; receive second data related to the tooth brushing activity from the first external electronic device; identify, based on the second data, a second tooth brushing pattern indicated by the tooth brushing activity; determine the similarity between the first tooth brushing pattern and the second tooth brushing pattern; and display guide information based on the first tooth brushing pattern based on determining that the similarity is less than a threshold level.

### CROSS-REFERENCE TO RELATED APPLICATIONS

This application is a continuation of International Application No. PCT/KR2024/018496 designating the United States, filed on Nov. 21, 2024, in the Korean Ministry of Intellectual Property Receiving Office and claiming priority to Korean Patent Application Nos. 10-2023-0164469, filed on Nov. 23, 2023, and 10-2024-0001530, filed on Jan. 4, 2024, in the Korean Ministry of Intellectual Property, the disclosures of each of which are incorporated by reference herein in their entireties.

### BACKGROUND

### Field

The disclosure relates to a personalized tooth brushing guide method, and an electronic device and a storage medium supporting the same.

### Description of Related Art

With the advancement of digital convergence in which various information and communication technologies are integrated, wearable devices linked to existing electronic devices and providing new types of functions and/or services have been proposed. These wearable devices can provide information related to user activity and/or user biometrics, from the view point that the wearable devices are operated as worn on or in close proximity to the user's body. For example, the wearable device can generate data related to user activity, based on its onboard sensors, and transmit the data to the linked electronic device, thereby supporting the provision of user activity information via various interfaces of the electronic device.

The above-described content may be provided as related art for the purpose of helping understanding of the disclosure. No assertion or determination is made as to whether the above-described content may be applied as prior art related to the disclosure.

### SUMMARY

An electronic device according to an example embodiment of the disclosure may include a wireless communication circuit, a display, at least one processor, comprising processing circuitry, and a memory for storing instructions.

According to an example embodiment of the disclosure, at least one processor, individually and/or collectively, may be configured to execute the instructions to cause the electronic device to: identify first data related to an electronic medical record of a user, based on detecting a coupling between a first external electronic device and a first object related to a tooth brushing activity of the user, determine a first tooth brushing pattern related to the tooth brushing activity of the user, based on the first data, receive second data related to the tooth brushing activity of the user from the first external electronic device via the wireless communication circuit, identify a second tooth brushing pattern indicated by the tooth brushing activity of the user, based on the second data, determine a similarity between the first tooth brushing pattern and the second tooth brushing pattern, and control the display to display guide information based on the first tooth brushing pattern, based on determining that the similarity is less than a specified threshold level.

A personalized tooth brushing guide method according to an example embodiment of the disclosure may include: identifying, by an electronic device, first data related to an electronic medical record of a user, based on detecting a coupling between a first external electronic device and a first object related to a tooth brushing activity of the user, determining, by the electronic device, a first tooth brushing pattern related to the tooth brushing activity of the user, based on the first data, receiving, by the electronic device, second data related to the tooth brushing activity of the user from the first external electronic device via a wireless communication circuit of the electronic device, identifying, by the electronic device, a second tooth brushing pattern indicated by the tooth brushing activity of the user, based on the second data, determining, by the electronic device, a similarity between the first tooth brushing pattern and the second tooth brushing pattern, and displaying guide information based on the first tooth brushing pattern, based on determining that the similarity is less than a specified threshold level.

In a non-transitory computer-readable storage medium on which a computer program which, when executed by at least one processor, comprising processing circuitry, individually and/or collectively, of an electronic device, cause the electronic device to perform operations comprising: identifying first data related to an electronic medical record of a user, based on detecting a coupling between a first external electronic device and a first object related to a tooth brushing activity of the user, determining, by the electronic device, a first tooth brushing pattern related to the tooth brushing activity of the user, based on the first data, receiving, by the electronic device, second data related to the tooth brushing activity of the user from the first external electronic device via a wireless communication circuit of the electronic device, identifying, by the electronic device, a second tooth brushing pattern indicated by the tooth brushing activity of the user, based on the second data, determining, by the electronic device, a similarity between the first tooth brushing pattern and the second tooth brushing pattern, and controlling, by the electronic device, a display of the electronic device to display guide information based on the first tooth brushing pattern, based on determining that the similarity is less than a specified threshold level.

An electronic device according to an example embodiment of the disclosure may include: a wireless communication circuit, a display, at least one processor, comprising processing circuitry, and a memory for storing instructions.

According to an example embodiment of the disclosure, at least one processor, individually and/or collectively, may be configured to execute the instructions to cause the electronic device to: identify a position of a first tooth for providing a tooth brushing pattern, based on first data about a dental treatment record, determine a first tooth brushing pattern corresponding to the identified position of the first tooth, and control the display to display at least one first user guide, based on the determined first tooth brushing pattern.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a diagram illustrating an example electronic device in a network environment according to various example embodiments.

FIG. 2 is a block diagram illustrating an example configuration of an electronic device according to various example embodiments.

FIG. 3 is a diagram illustrating a first external electronic device according to various example embodiments.

FIG. 4 is a flowchart illustrating an example operation of an electronic device of detecting a coupling between a first external electronic device and a first object, according to various example embodiments.

FIG. 5 is a diagram illustrating a coupled form of a first external electronic device and a first object, according to various example embodiments.

FIG. 6 is a diagram illustrating an example first user interface of an electronic device according to various example embodiments.

FIG. 7 is a flowchart illustrating an example operation of an electronic device of executing a tooth brushing guide service, according to various example embodiments.

FIG. 8 includes graphs illustrating data indicating a first tooth brushing pattern, according to various example embodiments.

FIG. 9 is a diagram illustrating an example first image indicating a user teeth condition displayed by an electronic device, according to various example embodiments.

FIG. 10 is a diagram illustrating an example second image for guiding a user's tooth brushing order displayed by an electronic device, according to various example embodiments.

FIG. 11 is a flowchart illustrating an example operation of an electronic device of detecting a coupling between a first external electronic device and a first object, according to various example embodiments.

FIG. 12 is a diagram illustrating an example second user interface of an electronic device according to various embodiments.

FIG. 13 is a flowchart illustrating an example operation of an electronic device of monitoring a tooth brushing activity of a user, according to various example embodiments.

FIG. 14 is a diagram illustrating an example third image for providing a feedback on a tooth brushing activity of a user displayed by an electronic device, according to various example embodiments.

FIG. 15 includes graphs illustrating data indicating a second tooth brushing pattern of a user, according to various example embodiments.

FIG. 16 is a flowchart illustrating an example operation of an electronic device of providing information on a tooth brushing activity of a user, according to various example embodiments.

FIG. 17 is a diagram illustrating an example third user interface of an electronic device, according to various example embodiments.

FIG. 18 is a flowchart illustrating an example personalized tooth brushing guide method according to various example embodiments.

In relation to the description of the drawings, the same or similar reference numerals may be used for the same or similar components.

### DETAILED DESCRIPTION

Various example embodiments of the disclosure are described below with reference to the accompanying drawings. However, this is not intended to limit the disclosure to a specific embodiment, and is to be understood as including various modifications, equivalents, and/or alternatives of the disclosure.

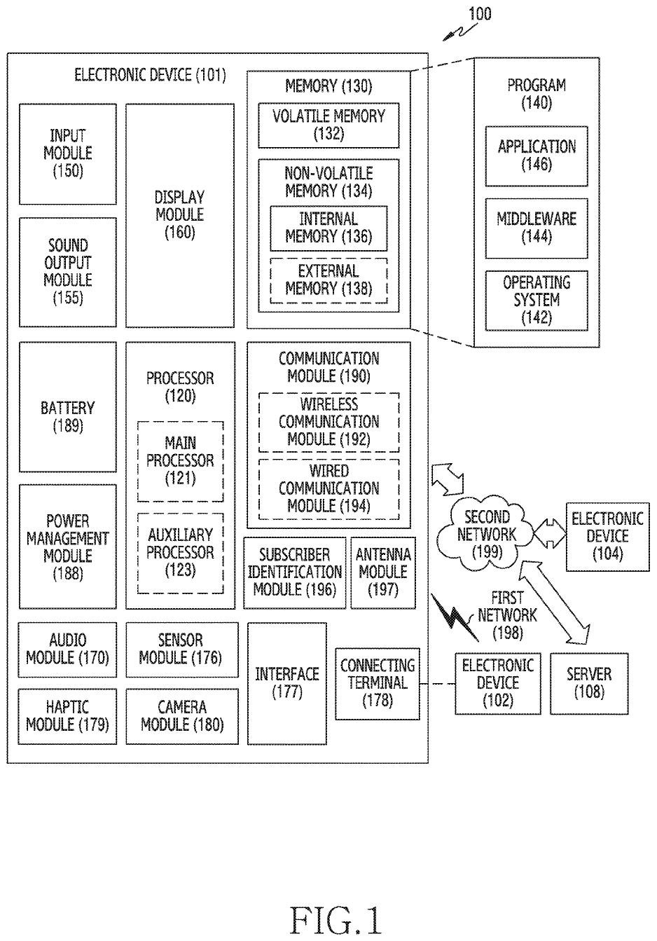

FIG. 1 is a block diagram illustrating an example electronic device 101 in a network environment 100 according to various example embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In various embodiments, at least one of the components (e.g., the connecting terminal 178) may be omitted from the electronic device 101, or one or more other components may be added in the electronic device 101. In various embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121. Thus, the processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

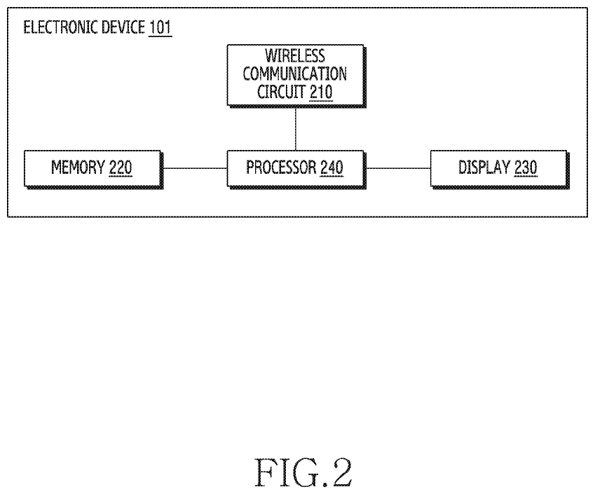

FIG. 2 is a block diagram illustrating an example configuration of an electronic device according to various example embodiments.

Referring to FIG. 2, an electronic device 101 of an embodiment (e.g., electronic device 101 of FIG. 1) may include a wireless communication circuit 210, a memory 220, a display 230, and a processor (e.g., including processing circuitry) 240. In various embodiments, the electronic device 101 may omit at least some of the components described above, or may further include other additional components. For example, the electronic device 101 may further include at least some of the components of the electronic device mentioned via FIG. 1.

In an embodiment, the wireless communication circuit 210 (e.g., wireless communication module 192 of FIG. 1) may support wireless communication between the electronic device 101 and at least one external electronic device. For example, the wireless communication circuit 210 may establish communication (or communication channel) of a defined communication protocol with the at least one external electronic device, and may transmit and receive data and/or a signal with the at least one external electronic device using the communication. According to an embodiment, the data may include data related to an electronic medical record of a user of the electronic device 101 and data related to a tooth brushing activity of the user.

In an embodiment, the memory 220 (e.g., memory 130 of FIG. 1) may store instructions related to functional operations of components of the electronic device 101. For example, the memory 220 may store instructions of, when executed by the processor 240, causing the components of the electronic device 101 to perform defined functional operations. In addition, the memory 220 may store data related to the operation of the electronic device 101. For example, the memory 220 may store, in the form of a database and/or table, data related to an electronic medical record of the user received from the at least one external electronic device via the wireless communication circuit. In addition, for example, the memory 220 may update and store the stored data according to a specified cycle or at the time of executing a specified application. In addition, the memory 220 may store at least one application related to a health service for the user of the electronic device 101. For example, the memory 220 may store at least one of an application installed in a preload form at the time of manufacturing the electronic device 101 and an application downloaded in a third party form from an online market.

In an embodiment, the display 230 (e.g., display module 160 of FIG. 1) may be at least partially exposed to the outside of the electronic device 101 and visually provide various contents. For example, the display 230 may receive a driving signal corresponding to image information from the processor 240 at a specified frame rate, process the driving signal, and display various graphical user interfaces provided by applications of the electronic device 101. In an embodiment, the graphical user interface may include content obtained (or generated) based on data related to an electronic medical record of the user of the electronic device 101 and/or data related to a tooth brushing activity of the user. In addition, the display 230 may receive various types of user inputs. For example, the display 230 may include a touch panel (or touch sensing circuit) in a portion of a layer of the display 230, and may detect a user input, based on the touch panel, and output a corresponding electrical signal.

In an embodiment, the processor 240 (e.g., processor 120 of FIG. 1) may include various processing circuitry and execute instructions (e.g., programming language code) stored in the memory 220 and perform overall control related to functional operations of the components of the electronic device 101. For example, the processor 240 may be electrically connected to the components of the electronic device 101, and transfer (or transmit) commands, signals, and/or data, which are involved when the components perform the functional operations, to the related components. The description of the processor 120 above applies equally to the processor 240 and the detailed description will not be repeated here.

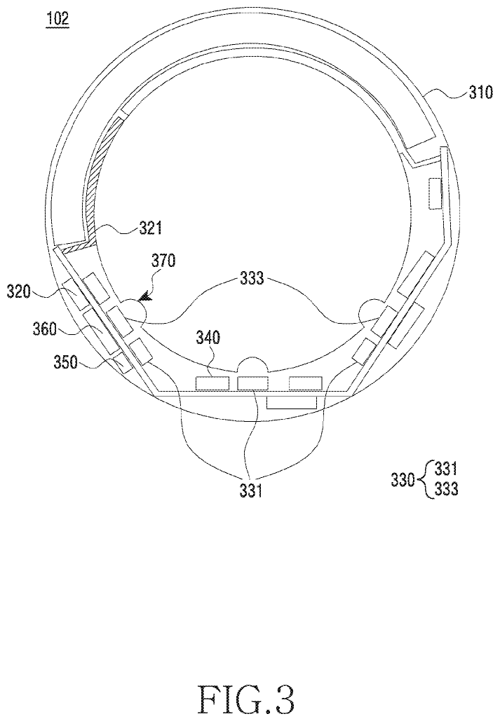

FIG. 3 is a diagram illustrating an example first external electronic device according to various example embodiments.

Referring to FIG. 3, a first external electronic device 102 (e.g., electronic device 102 of FIG. 1) of an embodiment may include a communication module (e.g., including communication circuitry) 320, an optical blood flow measurement sensor 330, an inertial sensor 340, a memory 350, and a processor (e.g., including processing circuitry) 360, which are arranged in an internal space of a housing 310. According to various embodiments, the first external electronic device 102 may omit at least some of the components described above, or may further include other additional components. For example, the first external electronic device 102 may further include a pressure sensor configured to detect the intensity of an external force applied to the housing 310.

In an embodiment, the first external electronic device 102 may be worn on the body (e.g., finger) of a user of an electronic device (e.g., electronic device 101 of FIG. 2) (or may get in contact with the user's body based on at least one protruding area 370 formed on the housing 310), and provide user's biometric information to the electronic device 101. For example, the processor 360 of the first external electronic device 102 may control the communication module 320 and an antenna 321, based on the execution of instructions stored in the memory 350, and transmit, to the electronic device 101, data and/or a signal regarding a user condition (e.g., biometric information) detected by a light-emitting device 331 and light-receiving device 333 of the optical blood flow measurement sensor 330. In this respect, the first external electronic device 102 may also be referred to as a wearable device.

In an embodiment, the first external electronic device 102 may be coupled to an object (e.g., toothbrush) related to a tooth brushing activity of the user, and may provide tooth brushing activity information of the user to the electronic device 101. For example, the processor 360 of the first external electronic device 102 may control the communication module 320 and the antenna 321, based on the execution of instructions stored in the memory 350, and transmit data and/or a signal regarding a motion, posture, and/or gesture of the first external electronic device 102 detected by the inertial sensor 340 (e.g., acceleration sensor and/or gyro sensor), to the electronic device 101. In addition, for example, the processor 360 of the first external electronic device 102 may control the communication module 320 and the antenna 321, and transmit data and/or a signal indicating the intensity (or pressure) of contact of the user's body (e.g., finger) against the first external electronic device 102, which is detected by the pressure sensor of the first external electronic device 102, to the electronic device 101.

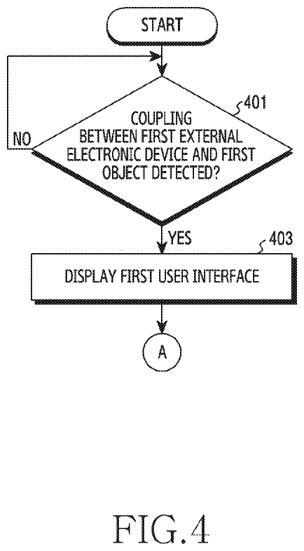

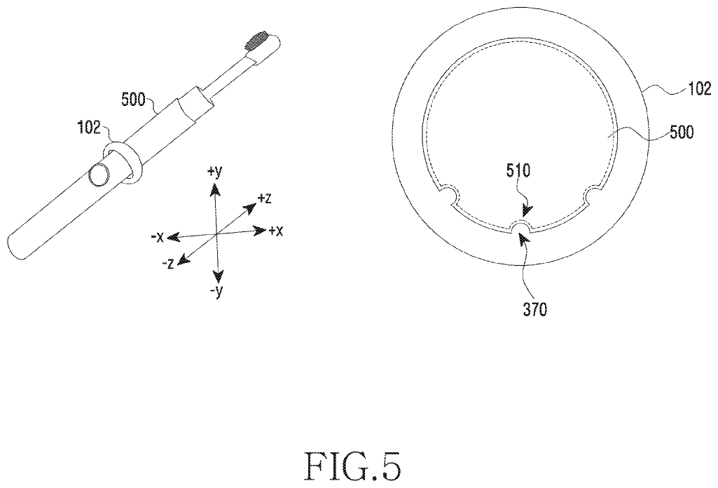

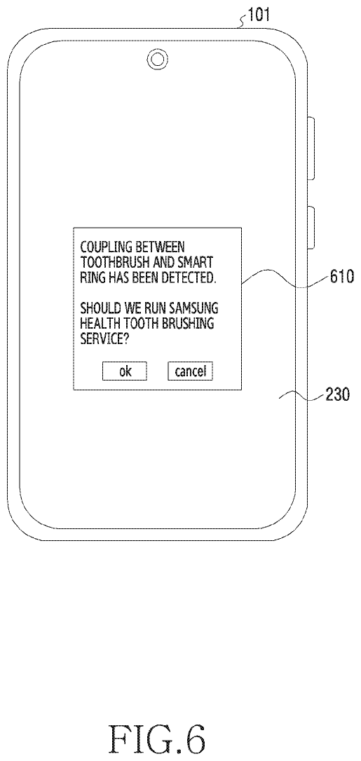

FIG. 4 is a flowchart illustrating an example operation of an electronic device of detecting a coupling between a first external electronic device and a first object, according to various example embodiments. FIG. 5 is a diagram illustrating an example coupled form of the first external electronic device and the first object, according to various example embodiments. FIG. 6 is a diagram illustrating an example first user interface of the electronic device according to various example embodiments.

Hereinafter, when FIG. 4 is described, FIGS. 5 and 6 may be referred to together. Operations of an electronic device mentioned in FIG. 4 may be performed sequentially, but may not necessarily be performed sequentially. For example, the order of the operations mentioned in FIG. 4 may be changed, or at least two operations may be performed in parallel.

Referring to FIGS. 4 and 5, in operation 401, an electronic device (e.g., electronic device 101 of FIG. 2) of an embodiment may determine whether a coupling between a first external electronic device 102 and a first object 500 has been detected. For example, a processor (e.g., processor 240 of FIG. 2) of the electronic device 101 may determine whether the first external electronic device 102 communicatively connected via a wireless communication circuit (e.g., wireless communication circuit 210 of FIG. 2) of the electronic device 101 has been coupled to the first object 500.

According to an embodiment, the first object 500 may include a toothbrush supporting a tooth brushing activity of a user, and the first object 500 may have a shape standardized with the first external electronic device 102. For example, when the first object 500 is viewed in a +Z direction or a −Z direction, at least one groove 510 may be formed in a housing of the first object 500, and may accommodate at least one protruding area 370 formed on a housing (e.g., housing 310 of FIG. 3) of the first external electronic device 102. Based on this, the first external electronic device 102 may be coupled to the first object 500 by accommodating the at least one protruding area 370 in the at least one groove 510 of the first object 500.

In an embodiment, the processor 240 of the electronic device 101 may determine that the first external electronic device 102 has been coupled to the first object 500, based on data and/or a signal received from the first external electronic device 102. For example, a processor (e.g., processor 360 of FIG. 3) of the first external electronic device 102 may transmit, to the electronic device 101, data and/or a signal indicating that the first external electronic device 102 has been coupled to the first object 500 rather than the user's body, based on the fact that no micro current is detected by an optical blood flow measurement sensor (e.g., optical blood flow measurement sensor 330 of FIG. 3), and the processor 240 of the electronic device 101 may determine a coupling between the first external electronic device 102 and the first object 500, based on the data and/or signal received from the first external electronic device 102. The processor 240 of the electronic device 101 may receive data and/or a signal generated by the optical blood flow measurement sensor 330 from the first external electronic device 102, and determine a coupling between the first external electronic device 102 and the first object 500, based on the fact that the signal and/or data do not indicate biometric information. For example, when the processor 360 of the first external electronic device 102 receives a user input to an input device (e.g., button and/or switch) formed in the housing of the first external electronic device 102, the processor 360 of the first external electronic device 102 may activate a specified mode (e.g., tooth brushing activity sensing mode) and transmit data and/or a signal indicating this to the electronic device 101, and the processor 240 of the electronic device 101 may determine a coupling between the first external electronic device 102 and the first object 500, based on the data and/or signal received from the first external electronic device 102.

According to various example embodiments, at least part of the first external electronic device 102 may be integrated with the first object 500. For example, the first external electronic device 102 may be included inside the housing of the first object 500. For example, at least some components (e.g., communication module 320 of FIG. 3, inertial sensor 340 of FIG. 3, and processor 360 of FIG. 3) of the first external electronic device 102 may be included inside the housing of the first object 500, and in this case, the at least some components of the first external electronic device 101 may be replaceable on the first object 500.

According to an embodiment, in operation 403, the processor 240 of the electronic device 101 may display a specified first user interface, based on the detection of the coupling between the first external electronic device 102 and the first object 500. Referring to FIG. 6, for example, the processor 240 of the electronic device 101 may execute a specified application (e.g., Samsung Health application), and control the display 230 to display a first user interface 610 via an execution screen of the application. According to an embodiment, the first user interface 610 may include content inquiring about whether to execute a tooth brushing care service via the application.

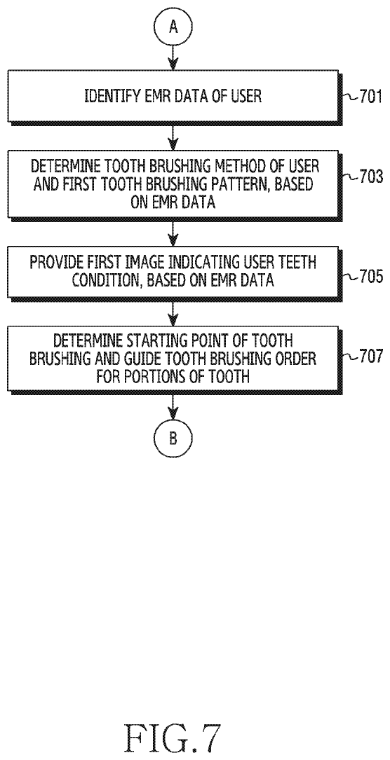

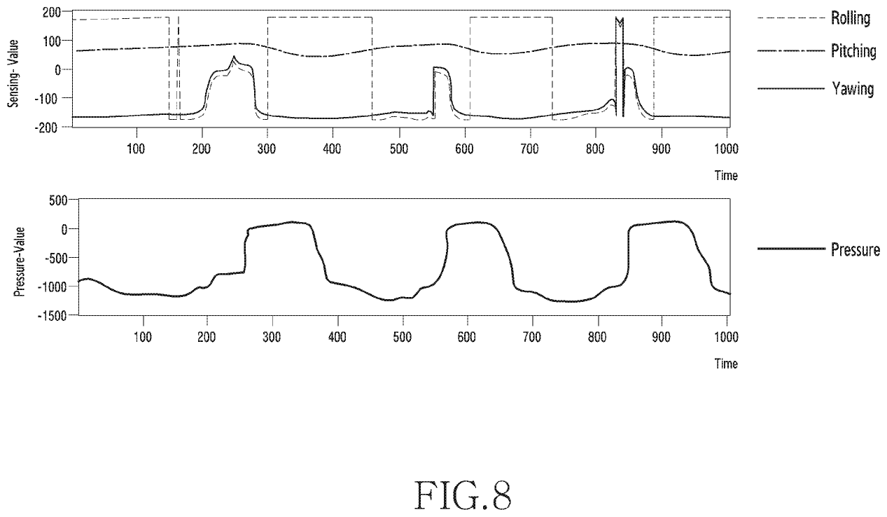

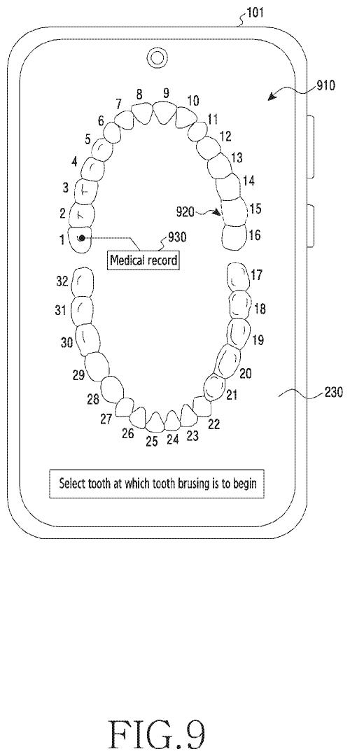

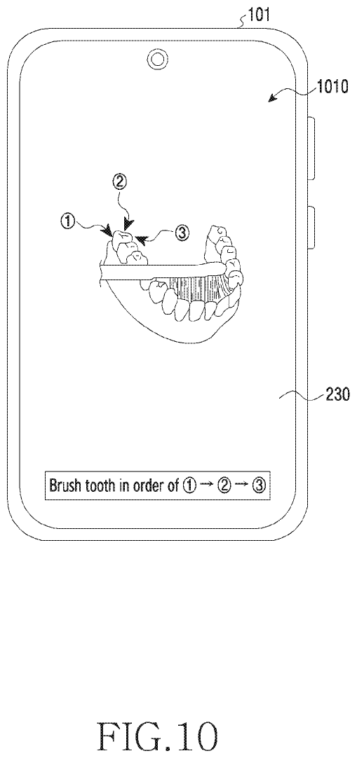

FIG. 7 is a flowchart illustrating an example operation of an electronic device of executing a tooth brushing guide service, according to various example embodiments. FIG. 8 includes graphs illustrating data indicating a first tooth brushing pattern, according to various example embodiments. FIG. 9 is a diagram illustrating an example first image indicating a user teeth condition displayed by an electronic device, according to various example embodiments. FIG. 10 is a diagram illustrating an example second image for guiding a user's tooth brushing order displayed by the electronic device, according to various example embodiments.

Hereinafter, when FIG. 7 is described, FIGS. 8, 9 and 10 may be referred to together. Operations of an electronic device mentioned in FIG. 7 may be performed sequentially, but may not necessarily be performed sequentially. For example, the order of the operations mentioned in FIG. 7 may be changed, or at least two operations may be performed in parallel.

Referring to FIG. 7, in operation 701, an electronic device (e.g., electronic device 101 of FIG. 2) of an embodiment may identify first data related to an electronic medical record of a user, based on executing a tooth brushing care service via an application. For example, a processor (e.g., processor 240 of FIG. 2) of the electronic device 101 may identify the first data that includes at least one of user identification information (e.g., name, gender, and/or age), image data (e.g., computed tomography (CT) and/or X-ray image data of a user's oral cavity), user teeth condition information (e.g., tooth count information, extracted tooth information, treated tooth information, dental procedure information, tooth-specific disease information, dentition information, and/or oral structure information), and expert comment information.

According to an embodiment, the processor 240 of the electronic device 101 may request first data related to the electronic medical record of the user from a second external electronic device (e.g., server 108 of FIG. 1) that is based on a fast healthcare interoperability resource, and obtain the first data from the second external electronic device 108 via a wireless communication circuit (e.g., wireless communication circuit 210 of FIG. 2), thereby identifying the first data. The processor 240 of the electronic device 101 may receive first data from the second external electronic device 108 in an initial setup operation of a tooth brushing care service, store the first data in a memory (e.g., memory 220 of FIG. 2) of the electronic device 101, and load the first data stored in the memory 220 at the time of executing the tooth brushing care service, thereby identifying the first data. The processor 240 of the electronic device 101 may request and obtain first data from the second external electronic device 108, update first data previously stored in the memory 220, based on the obtained first data, and load the updated first data from the memory 220 at the time of executing the tooth brushing care service, thereby identifying the first data.

According to an embodiment, in operation 703, the processor 240 of the electronic device 101 may determine a user's tooth brushing method and a first tooth brushing pattern corresponding to the tooth brushing method, based on identifying the first data related to the electronic medical record of the user. Table 1 and FIG. 8 may be referred with respect to the tooth brushing method and the tooth brushing pattern.

TABLE 1

| Age | Teeth condition | Tooth brushing Method |
|---|---|---|
| 5-9 | Normal | Fones, Scrubbing |
| 10+ | Normal | Roll |
| | Stomatitis | Tooth-pick |
| | Periodontitis | Bass |
| | Dental braces | Charter's |
| | Implant | Charter's |

Table 1 shows a tooth brushing method recommended (or appropriate) for a tooth brushing activity depending on various parameters (e.g., age and/or teeth condition). For example, when a specific user is 10 years or older and has a normal teeth condition, a rolling tooth brushing method is recommended for the corresponding user. According to various embodiments, the tooth brushing method described in Table 1 is merely an example, and the recommended tooth brushing method may vary depending on a combination of various parameters. Referring to FIG. 8, when each of teeth of different positions is brushed in a tooth brushing method recommended for a specific user using a toothbrush (or first external electronic device 102 coupled to the first object 500 of FIG. 5 according to an embodiment) capable of detecting a tooth brushing activity, a specific tooth brushing pattern is modeled for each tooth. For example, the tooth brushing pattern modeled for the specific tooth is indicated as data including a unique change of at least one of a roll value, a pitch value, a yaw value, and a pressure value with the lapse of time, when a plurality of portions of the specific tooth (e.g., outer surface of the tooth, an upper or lower surface of the tooth, and an inner surface of the tooth) are brushed in a predefined order.

In an embodiment, information about the tooth brushing method of the user of the electronic device 101 and the first tooth brushing pattern corresponding to the tooth brushing method may be included in the first data related to the electronic medical record of the user that the electronic device 101 receives from the second external electronic device 108. Based on this, when obtaining the first data from the second external electronic device 108 or the memory 220 of the electronic device 101 and identifying the first data, the processor 240 of the electronic device 101 may determine a tooth brushing method corresponding to identification information and teeth condition information of the user of the electronic device 101, based on the first data, and determine a first tooth brushing pattern of the determined tooth brushing method for each of teeth of different positions of the user. Alternatively, the information about the tooth brushing method of the user of the electronic device 101 and the first tooth brushing pattern corresponding to the tooth brushing method may be omitted from the first data related to the electronic medical record of the user that the electronic device 101 receives from the second external electronic device 108. In this case, when obtaining the first data from the second external electronic device 108 or the memory 220 of the electronic device 101 and identifying the first data, the processor 240 of the electronic device 101 may apply the first data to a big data-based algorithm and determine the user's tooth brushing method and the first tooth brushing pattern.

According to an embodiment, in operation 705, the processor 240 of the electronic device 101 may provide a first image indicating a user teeth condition, based on obtaining the first data from the second external electronic device 108 or the memory 220 of the electronic device 101 and identifying the first data. Referring to FIG. 9, for example, the processor 240 of the electronic device 101 may generate a first image 910 by rendering image data (e.g., CT and/or X-ray image data of a user's oral cavity) included in the first data, processing at least part of the image data, or modeling an image based on information included in the first data, and control the display 230 to display the first image 910 on an execution screen of an application executing a tooth brushing care service. According to an embodiment, the first image 910 may indicate a unique teeth condition of the user of the electronic device 101. For example, the processor 240 of the electronic device 101 may generate the first image 910 such that the first image 910 indicates at least one of a user's dentition, the number of teeth, and an oral structure, which are identified based on the first data. In addition, for example, the processor of the electronic device 101 may generate the first image 910 such that an extracted tooth of the user identified based on the first data is displayed on the first image 910 with a specified effect 920 (e.g., dotted-line processing of an outline of the extracted tooth). In addition, for example, the processor 240 of the electronic device 101 may identify a treated tooth of the user, based on the first data, and display content 930, which is selectable by a user input, in an area corresponding to the treated tooth on the first image 910. The processor 240 of the electronic device 101 may provide (e.g., display through a pop-up window) medical record information and/or expert comment information about a corresponding tooth, based on receiving a user input for the content 930.

According to an embodiment, in operation 707, the processor 240 of the electronic device 101 may determine a point (e.g., tooth of a specific position) at which a tooth brushing activity of the user is to begin, and guide a tooth brushing order for portions of the tooth. For example, the processor 240 of the electronic device 101 may display content requesting for selecting a tooth at which the tooth brushing activity of the user is to begin, on the first image 910, and in response to the request, receive a user input of selecting a specific tooth (e.g., tooth of code 1) from the first image 910.

In an embodiment, when the tooth at which the tooth brushing activity of the user is to begin is determined based on receiving the user input of selecting the specific tooth, the processor 240 of the electronic device 101 may control the display 230 to display a second image for guiding a tooth brushing order. Referring to FIG. 10, for example, the processor 240 of the electronic device 101 may generate a second image 1010 indicating a unique teeth condition of the user, based on the first data obtained and identified from the second external electronic device 108 or the memory 220 of the electronic device 101, and control the display 230 to display the second image 1010. According to an embodiment, the processor 240 of the electronic device 101 may display content for guiding a tooth brushing order corresponding to a first tooth brushing pattern, on the second image 1010. For example, the processor 240 of the electronic device 101 may display content for guiding the user to perform tooth brushing in a tooth brushing order corresponding to the change of roll, pitch, and yaw values indicated by data of the first tooth brushing pattern. For example, when the data of the first tooth brushing pattern indicates the change of the roll, pitch, and yaw values that are generated when the user brushes the tooth in a tooth brushing order of an outer surface, upper or lower surface, and inner surface of the tooth, the processor 240 may display content for guiding the tooth brushing order of the outer surface (\textcircled{1}), upper or lower surface (\textcircled{2}), and inner surface (\textcircled{3}) of the tooth at which the tooth brushing activity is to begin.

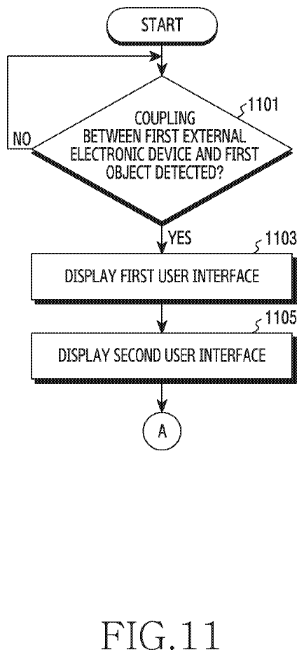

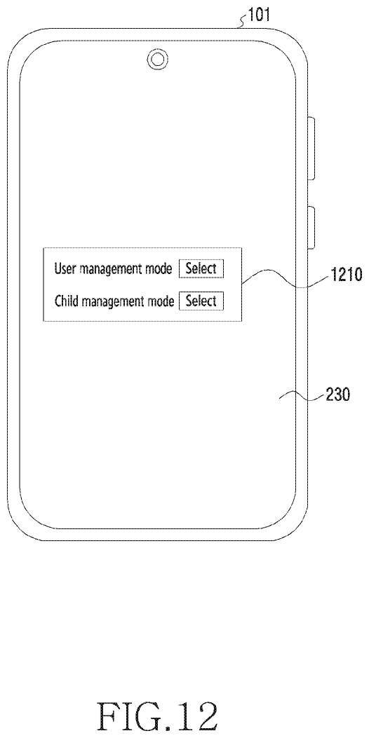

FIG. 11 is a flowchart illustrating an example operation of an electronic device of detecting a coupling between a first external electronic device and a first object, according to various embodiments. FIG. 12 is a diagram illustrating an example second user interface of an electronic device according to various example embodiments.

Hereinafter, when FIG. 11 is described, FIG. 12 may be referred to together. Operations of an electronic device mentioned in FIG. 11 may be performed sequentially, but may not necessarily be performed sequentially. For example, the order of the operations mentioned in FIG. 11 may be changed, or at least two operations may be performed in parallel. At least some of the operations of the electronic device mentioned via FIG. 11 may be the same as or similar to the operations of the electronic device described through the preceding drawings, and a repeated description of the same as or similar operation may not be repeated here.

Referring to FIG. 11, in operation 1101, an electronic device (e.g., electronic device 101 of FIG. 2) of an embodiment may determine whether a coupling between a first external electronic device (e.g., first external electronic device 102 of FIG. 5) and a first object (e.g., first object 500 of FIG. 5) has been detected. For example, a processor (e.g., processor 240 of FIG. 2) of the electronic device 101 may determine whether the first external electronic device 102 has been coupled to the first object 500, based on data and/or a signal received from the first external electronic device 102.

In an embodiment, in operation 1103, the processor 240 of the electronic device 101 may control a display (e.g., display 230 of FIG. 6) to display a specified first user interface (e.g., first user interface 610 of FIG. 6), based on detecting the coupling between the first external electronic device 102 and the first object 500. For example, the processor 240 of the electronic device 101 may execute a specified application (e.g., Samsung Health application), and display the first user interface 610 including content inquiring about whether to execute a tooth brushing care service via an execution screen of the application.

In an embodiment, in operation 1105, the processor 240 of the electronic device 101 may display a specified second user interface, based on receiving a user input of instructing the execution of the tooth brushing care service. Referring to FIG. 12, for example, the processor 240 of the electronic device 101 may control the display 230 to display a second user interface 1210 via the execution screen of the application. According to an embodiment, the second user interface 1210 may include content capable of selecting one of a plurality of modes (e.g., user mode and child mode) related to the tooth brushing care service, and the plurality of modes may be referred in order to identify by the processor 240 of the electronic device 101 a user who performs a tooth brushing activity.

According to various embodiments, the processor 240 of the electronic device 101 may also perform the operations of FIG. 7 described above, based on the mode (e.g., user mode or child mode) selected by the user input. For example, when the processor 240 receives a user input of selecting a first mode (e.g., child mode), in operation 701, the processor 240 may obtain first data related to an electronic medical record of an external user (e.g., child) corresponding to the first mode from a second external electronic device (e.g., server 108 of FIG. 1) or a memory of the electronic device 101 (e.g., memory 220 of FIG. 2), and identify the first data. In addition, for example, in operations 705 and 707, the processor 240 of the electronic device 101 may generate a first image and second image indicating a teeth condition of the external user, based on the first data related to the external user. According to various embodiments, the processor 240 of the electronic device 101 may control the display (e.g., display 230 of FIG. 2) to display the first image and the second image, or control the display 230 to display the first image and the second image in reverse, in consideration of a scenario in which the user performs a tooth brushing activity for the external user (e.g., scenario in which the user (parent) brushes teeth of the external user (child)).

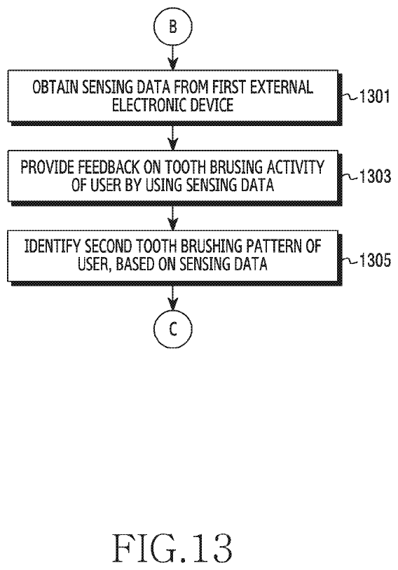

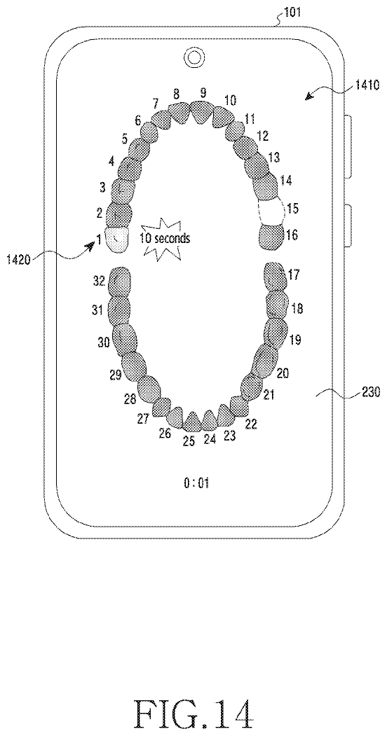

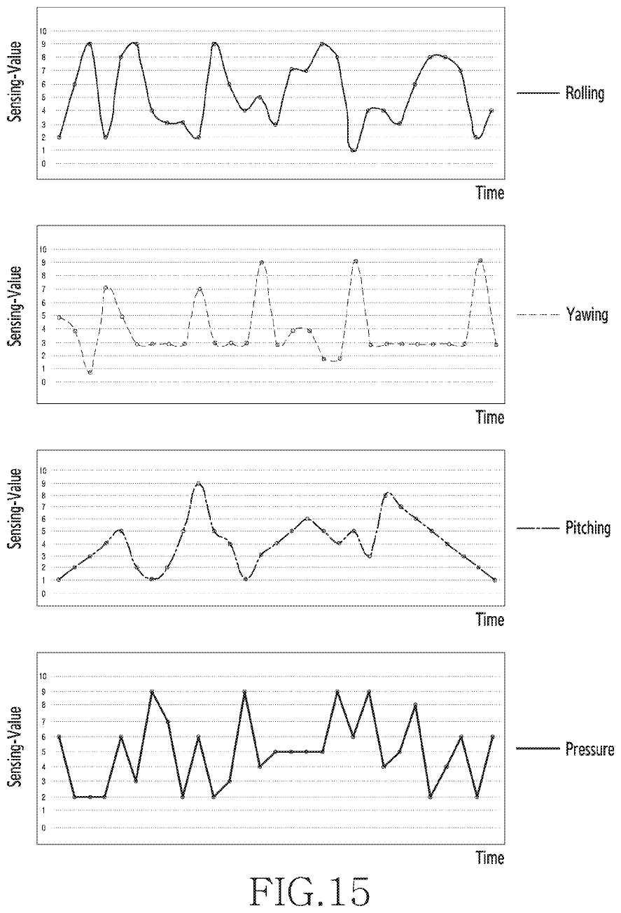

FIG. 13 is a flowchart illustrating an example operation of an electronic device of monitoring a tooth brushing activity of a user, according to various example embodiments. FIG. 14 is a diagram illustrating an example third image for providing a feedback on the tooth brushing activity of the user displayed by the electronic device, according to various example embodiments. FIG. 15 includes graphs illustrating data indicating a second tooth brushing pattern of the user, according to various example embodiments.

Hereinafter, when FIG. 13 is described, FIGS. 14 and 15 may be referred to together. Operations of an electronic device mentioned in FIG. 13 may be performed sequentially, but may not necessarily be performed sequentially. For example, the order of the operations mentioned in FIG. 13 may be changed, or at least two operations may be performed in parallel.

Referring to FIG. 13, in operation 1301, an electronic device (e.g., electronic device 101 of FIG. 2) of an embodiment may obtain sensing data from a first external electronic device (e.g., first external electronic device 102 of FIG. 5) coupled to a first object (e.g., first object 500 of FIG. 5). The sensing data may be referred as second data. For example, the processor 240 of the electronic device 101 may receive second data about a motion, posture, and/or gesture of the first external electronic device 102, which are detected by an inertial sensor (e.g., inertial sensor 340 of FIG. 3 including an acceleration sensor and/or a gyro sensor) of the first external electronic device 102 depending on a tooth brushing activity of a user, from the first external electronic device 102 via a wireless communication circuit (e.g., wireless communication circuit 210 of FIG. 2). For example, the processor 240 of the electronic device 101 may receive second data indicating the intensity of contact of the user's body against the first external electronic device 102, which is detected by a pressure sensor of the first external electronic device 102 depending on the tooth brushing activity of the user, from the first external electronic device 102 via the wireless communication circuit 210.

According to an embodiment, in operation 1303, the processor 240 of the electronic device 101 may provide a feedback on the tooth brushing activity of the user, based on receiving the sensing data from the first external electronic device 102. In this regard, referring to FIG. 14, the processor 240 of the electronic device 101 may generate a third image 1410 for which a specified effect (e.g., displaying user teeth in a specified first level of color) is applied to a first image (e.g., first image 910 of FIG. 9) indicating a user teeth condition in order to provide a feedback on the tooth brushing activity of the user, and may control the display 230 to display the third image 1410. According to an embodiment, based on the sensing data received from the first external electronic device 102, the processor 240 of the electronic device 101 may identify a tooth (e.g., tooth of code 1) on which the tooth brushing activity of the user is being performed, and the intensity of pressure of the user's body applied to the first external electronic device 101 so as to brush the corresponding tooth. In an embodiment, the processor 240 of the electronic device 101 may display the tooth on which the tooth brushing activity of the user is being performed in the third image 1410, with a specified effect 1420, based on the identified tooth and the pressure intensity associated with the corresponding tooth. For example, the processor 240 of the electronic device 101 may display the tooth on which the tooth brushing activity is being performed, in a second level of color that is lower (or lighter) than the specified first level of color on the third image 1410. In addition, for example, the processor 240 of the electronic device 101 may display the tooth on which the tooth brushing activity is being performed, in a stepwisely lower level of color on the third image 1410, with the lapse of a specified tooth brushing time.

According to an embodiment, in operation 1305, the processor 240 of the electronic device 101 may identify a second tooth brushing pattern of the user, based on the sensing data received from the first external electronic device 102. For example, the processor 240 of the electronic device 101 may identify the second tooth brushing pattern for the tooth on which the tooth brushing activity of the user is being performed (or has been performed), based on the second data, while being providing the feedback on the tooth on which the tooth brushing activity of the user is being performed or after completing the provision of the feedback on the tooth. In this regard, referring to FIG. 15, the sensing data received from the first external electronic device 102 while the tooth brushing activity of the user for the specific tooth is being performed may indicate the change of at least one of a roll value, a pitch value, a yaw value, and a pressure value that are detected by the first external electronic device 101 while the specific tooth is being brushed. In an embodiment, the processor 240 of the electronic device 101 may model the change of at least one of the roll value, pitch value, yaw value, and pressure value indicated by (or included in) the sensing data, thereby identifying the second tooth brushing pattern for the tooth on which the tooth brushing activity of the user is being performed (or has been performed).

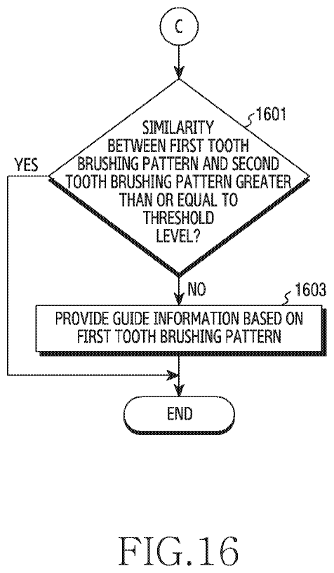

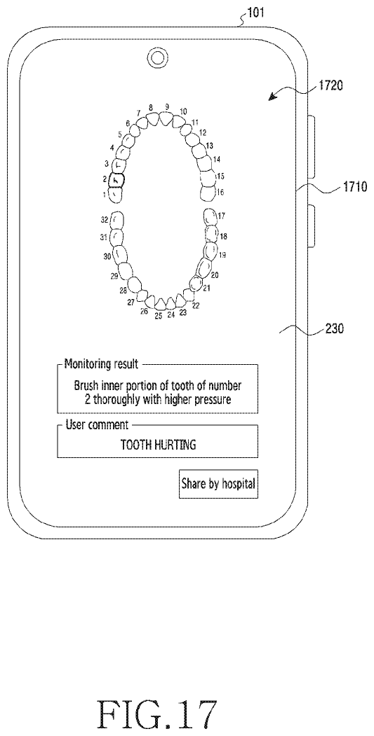

FIG. 16 is a flowchart illustrating an example operation of an electronic device of providing user's tooth brushing activity information, according to various example embodiments. FIG. 17 is a diagram illustrating an example third user interface of the electronic device according to various example embodiments.

Hereafter, when FIG. 16 is described, FIG. 17 may be referred to together. Operations of an electronic device mentioned in FIG. 16 may be performed sequentially, but may not necessarily be performed sequentially. For example, the order of the operations mentioned in FIG. 16 may be changed, or at least two operations may be performed in parallel.

Referring to FIG. 16, in operation 1601, an electronic device (e.g., electronic device 101 of FIG. 2) of an embodiment may determine whether a similarity between a first tooth brushing pattern and a second tooth brushing pattern is greater than or equal to a specified threshold level. For example, a processor (e.g., processor 240 of FIG. 2) of the electronic device 101 may compare a first tooth brushing pattern for a specific tooth of a tooth brushing method determined for a user, with a second tooth brushing pattern identified based on second data received from a first external electronic device (e.g., first external electronic device 102 of FIG. 5) during a tooth brushing activity of the user for the corresponding tooth. According to an embodiment, the processor 240 of the electronic device 101 may compare the change of the first tooth brushing pattern (e.g., change of a roll value, pitch value, yaw value, and pressure value indicated by data of the first tooth brushing pattern), with the change of the second tooth brushing pattern (e.g., change of a roll value, pitch value, yaw value, and pressure value indicated by data of the second tooth brushing pattern), and determine the similarity, based on a difference of the comparison result between the first tooth brushing pattern and the second tooth brushing pattern. According to various embodiments, when determining the similarity between the first tooth brushing pattern and the second tooth brushing pattern for the specific tooth, the processor 240 may determine a comprehensive similarity for a plurality of portions of the specific tooth (e.g., outer surface of the tooth, upper or lower surface of the tooth, and inner surface of the tooth), or determine an individual similarity for each of the plurality of portions.

In an embodiment, based on determining that the similarity between the first tooth brushing pattern and the second tooth brushing pattern is smaller than the specified threshold level, in operation 1603, the processor 240 of the electronic device 101 may provide guide information that is based on the first tooth brushing pattern. Referring to FIG. 17, for example, the processor 240 of the electronic device 101 may control the display 230 to display a third user interface 1710 for providing the guide information on an execution screen of an application executing a tooth brushing care service. According to an embodiment, the third user interface 1710 may include a first image 1720 indicating a user teeth condition, and content indicating a monitoring result of a tooth brushing activity of a user. For example, the processor 240 of the electronic device 101 may display content for guiding a tooth brushing method for a tooth (e.g., tooth of code 2) for which a similarity between the first tooth brushing pattern and the second tooth brushing pattern is determined to be smaller than the specified threshold level or portion of the tooth (e.g., inner portion of the tooth of code 2) for which the similarity is determined to be smaller than the specified threshold level, in the third user interface 1710, such that the second tooth brushing pattern identified during the tooth brushing activity of the user for the corresponding tooth may indicate data (e.g., change of a roll value, pitch value, yaw value, and pressure value) corresponding to the first tooth brushing pattern determined for the corresponding tooth. According to various embodiments, the processor 240 of the electronic device 101 may further display an input area capable of inputting user content to the third user interface 1710 and content capable of transmitting information about the third user interface 1710 to a medical institution (e.g., server of the medical institution) linked to the electronic device 101.

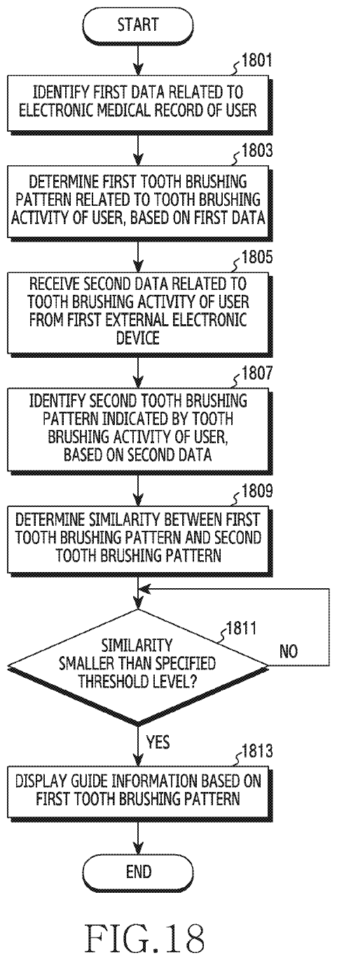

FIG. 18 is a flowchart illustrating an example personalized tooth brushing guide method according to various example embodiments.

Hereinafter, at least some of operations of an electronic device of FIG. 18 may be the same as or similar to the operations of the electronic device described through the preceding drawings, and a repeated description may not be repeated below. The operations of the electronic device mentioned in FIG. 18 may be performed sequentially, but may not necessarily be performed sequentially. For example, the order of the operations mentioned in FIG. 18 may be changed, or at least two operations may be performed in parallel.

Referring to FIG. 18, in operation 1801, an electronic device (e.g., electronic device 101 of FIG. 2) of an embodiment may identify first data related to an electronic medical record of a user of the electronic device 101. For example, the electronic device 101 may request and obtain first data from a second external electronic device (e.g., server 108 of FIG. 1), thereby identifying the first data. The electronic device 101 may load first data previously stored in a memory (e.g., memory 220 of FIG. 2), thereby identifying the first data. The electronic device 101 may update first data previously stored in the memory 220, based on the first data obtained from the second external electronic device 108, and load the updated first data from the memory 220, thereby identifying the first data.

In operation 1803, the electronic device 101 of an embodiment may determine a first tooth brushing pattern related to a tooth brushing activity of the user, based on the identified first data. For example, the electronic device 101 may determine a tooth brushing method corresponding to user's identification information and teeth condition information, based on the first data, and determine a first tooth brushing pattern of the determined tooth brushing method for each of user teeth of different positions. In an embodiment, the first tooth brushing pattern may include data indicating a unique change of at least one of a roll value, a pitch value, a yaw value, and a pressure value with the lapse of time, when portions of each of the teeth of different positions (e.g., outer surface of the tooth, upper or lower surface of the tooth, and inner surface of the tooth) are brushed in a predefined order using the determined tooth brushing method.

In operation 1805, the electronic device 101 of an embodiment may receive second data related to a tooth brushing activity of the user from a first external electronic device (e.g., first external electronic device 102 of FIG. 5) coupled to a first object (e.g., first object 500 of FIG. 5). For example, the electronic device 101 may receive second data about a motion, posture, and/or gesture of the first external electronic device 102 that are detected by a sensor of the first external electronic device 102 depending on the tooth brushing activity of the user. In addition, the electronic device 101 may receive second data indicating the intensity of contact of the user's body against the first external electronic device 102 that is detected by the sensor of the first external electronic device 102 depending on the tooth brushing activity of the user.

In operation 1807, the electronic device 101 of an embodiment may identify a second tooth brushing pattern indicated by the tooth brushing activity of the user, based on the second data. For example, the electronic device 101 may model the change of at least one of a roll value, pitch value, yaw value, and pressure value indicated by the second data for a tooth on which the tooth brushing activity of the user is being performed (or has been performed), thereby identifying the second tooth brushing pattern for the corresponding tooth.

In operations 1809 and 1811, the electronic device 101 of an embodiment may determine a similarity between the first tooth brushing pattern and the second tooth brushing pattern. For example, the electronic device 101 may compare a first tooth brushing pattern of a tooth brushing method determined for a specific tooth, with a second tooth brushing pattern identified based on the second data received from the first external electronic device 102 during the tooth brushing activity of the user for the corresponding tooth, thereby determining whether the similarity between the first tooth brushing pattern and the second tooth brushing pattern is smaller than a specified threshold level. According to various embodiments, the similarity between the first tooth brushing pattern and the second tooth brushing pattern may be determined comprehensively for a plurality of portions of the specific tooth (e.g., outer surface of the tooth, upper or lower surface of the tooth, and inner surface of the tooth), or may be determined individually for each of the plurality of portions.

In operation 1813, the electronic device 101 of an embodiment may display guide information that is based on the first tooth brushing pattern. For example, the electronic device 101 may display content indicating a monitoring result of the tooth brushing activity of the user via the display, based on the determination that the similarity between the first tooth brushing pattern and the second tooth brushing pattern is smaller than the specified threshold level. According to an embodiment, the content may include content for guiding a tooth brushing method for a tooth (or portion of the tooth) determined to have the similarity smaller than the specified threshold level, such that a second tooth brushing pattern identified when the corresponding tooth is brushed may indicate data (e.g., change of a roll value, pitch value, yaw value, and pressure value) corresponding to a first tooth brushing pattern determined for the corresponding tooth.

An electronic device of an example embodiment of the disclosure may include a wireless communication circuit, a display, a processor, and a memory for storing instructions.

According to an example embodiment of the disclosure, the instructions may be executed by the processor to cause the electronic device to identify first data related to an electronic medical record of a user, based on detecting a coupling between a first external electronic device and a first object related to a tooth brushing activity of the user, determine a first tooth brushing pattern related to the tooth brushing activity of the user, based on the first data, receive second data related to the tooth brushing activity of the user from the first external electronic device via the wireless communication circuit, identify a second tooth brushing pattern indicated by the tooth brushing activity of the user, based on the second data, determine a similarity between the first tooth brushing pattern and the second tooth brushing pattern, and control the display to display guide information that is based on the first tooth brushing pattern, based on determining that the similarity is smaller than a specified threshold level.

According to an example embodiment of the disclosure, the instructions may be executed by the processor to cause the electronic device to, based on obtaining the first data, control the display to display a first image indicating a user teeth condition, based on the first data.

According to an example embodiment of the disclosure, the instructions may be executed by the processor to cause the electronic device to determine a tooth corresponding to a position where the tooth brushing activity of the user is to begin, based on a user input on the first image.

According to an example embodiment of the disclosure, the instructions may be executed by the processor to cause the electronic device to control the display to display a second image for guiding a tooth brushing order corresponding to the first tooth brushing pattern for portions of a tooth of the user.

According to an example embodiment of the disclosure, the instructions may be executed by the processor to cause the electronic device to control the display to display content for identifying the user performing the tooth brushing activity, based on detecting the coupling between the first external electronic device and the first object.

According to an example embodiment of the disclosure, the second data may indicate the change of at least one of a roll value, a pitch value, a yaw value, and a pressure value that are detected by the first external electronic device coupled to the first object.

According to an example embodiment of the disclosure, the instructions may be executed by the processor to cause the electronic device to control the display to display a third image to which a specified effect generated based on the first image is applied.

According to an example embodiment of the disclosure, the specified effect may include an effect of stepwisely changing a color of a tooth on which the tooth brushing activity of the user is being performed, based on a progress of the tooth brushing activity of the user.

According to an example embodiment of the disclosure, the first external electronic device may include a wearable device that, when decoupled from the first object, is worn on the user's body and provides biometric information of the user to the electronic device.

A personalized tooth brushing guide method according to an example embodiment of the disclosure may include identifying, by an electronic device, first data related to an electronic medical record of a user, based on detecting a coupling between a first external electronic device and a first object related to a tooth brushing activity of the user, determining, by the electronic device, a first tooth brushing pattern related to the tooth brushing activity of the user, based on the first data, receiving, by the electronic device, second data related to the tooth brushing activity of the user from the first external electronic device via a wireless communication circuit of the electronic device, identifying, by the electronic device, a second tooth brushing pattern indicated by the tooth brushing activity of the user, based on the second data, determining, by the electronic device, a similarity between the first tooth brushing pattern and the second tooth brushing pattern, and controlling, by the electronic device, a display of the electronic device to display guide information that is based on the first tooth brushing pattern, based on determining that the similarity is smaller than a specified threshold level.

According to an example embodiment of the disclosure, the operation of obtaining the first data may include the operation of, based on obtaining the first data, controlling the display to display a first image indicating a user teeth condition, based on the first data.

According to an example embodiment of the disclosure, the operation of displaying the first image may include the operation of determining a tooth corresponding to a position where the tooth brushing activity of the user is to begin, based on a user input on the first image.

According to an example embodiment of the disclosure, the operation of determining the first tooth brushing pattern may include the operation of controlling the display to display a second image for guiding a tooth brushing order corresponding to the first tooth brushing pattern for portions of a tooth of the user.

According to an example embodiment of the disclosure, the method may further include the operation of controlling the display to display content for identifying the user performing the tooth brushing activity, based on detecting the coupling between the first external electronic device and the first object.

According to an example embodiment of the disclosure, the second data may indicate the change of at least one of a roll value, a pitch value, a yaw value, and a pressure value that are detected by the first external electronic device coupled to the first object.

According to an example embodiment of the disclosure, the operation of displaying the first image may include the operation of controlling the display to display a third image to which a specified effect generated based on the first image is applied.

According to an example embodiment of the disclosure, the specified effect may include an effect of stepwisely changing a color of a tooth on which the tooth brushing activity of the user is being performed, based on a progress of the tooth brushing activity of the user.

According to an example embodiment of the disclosure, the first external electronic device may include a wearable device that, when decoupled from the first object, is worn on the user's body and provides biometric information of the user to the electronic device.

In a non-transitory computer-readable storage medium on which a computer program for executing a personalized tooth brushing guide method of an example embodiment of the disclosure is recorded, the computer program may, when executed by an electronic device, cause the electronic device to perform the operations of identifying, by an electronic device, first data related to an electronic medical record of a user, based on detecting a coupling between a first external electronic device and a first object related to a tooth brushing activity of the user, determining, by the electronic device, a first tooth brushing pattern related to the tooth brushing activity of the user, based on the first data, receiving, by the electronic device, second data related to the tooth brushing activity of the user from the first external electronic device via a wireless communication circuit of the electronic device, identifying, by the electronic device, a second tooth brushing pattern indicated by the tooth brushing activity of the user, based on the second data, determining, by the electronic device, a similarity between the first tooth brushing pattern and the second tooth brushing pattern, and controlling, by the electronic device, a display of the electronic device to display guide information that is based on the first tooth brushing pattern, based on determining that the similarity is smaller than a specified threshold level.

According to an example embodiment of the disclosure, the operation of obtaining the first data may include the operation of, based on obtaining the first data, controlling the display to display a first image indicating a user teeth condition, based on the first data.

According to an example embodiment of the disclosure, the operation of displaying the first image may include the operation of determining a tooth corresponding to a position where the tooth brushing activity of the user is to begin, based on a user input on the first image.

According to an example embodiment of the disclosure, the operation of determining the first tooth brushing pattern may include the operation of controlling the display to display a second image for guiding a tooth brushing order corresponding to the first tooth brushing pattern for portions of a tooth of the user.

An electronic device according to an example embodiment of the disclosure may include a wireless communication circuit, a display, a processor, and a memory for storing instructions.

According to an example embodiment of the disclosure, when executed by the processor, the instructions may cause the electronic device to identify a position of a first tooth for providing a tooth brushing pattern, based on first data about a dental treatment record, determine a first tooth brushing pattern corresponding to the identified position of the first tooth, and control the display to provide at least one first user guide, based on the determined first tooth brushing pattern.

According to an example embodiment of the disclosure, the first tooth brushing pattern may include at least one of a rolling including an operation of moving a toothbrush from top to bottom or from bottom to top of the first tooth, a pitching including an operation of moving the toothbrush from left to right or from right to left of the first tooth, or a pressing including an operation of pressing the toothbrush against the first tooth.

According to an example embodiment of the disclosure, when executed by the processor, the instructions cause the electronic device to receive second data about a second tooth brushing pattern indicated by a tooth brushing activity of a user from a first external electronic device, and control the display to provide the at least one first user guide, based on a comparison between the first tooth brushing pattern and the second tooth brushing pattern.

According to an example embodiment of the disclosure, the first tooth may include at least one or more teeth for which the first tooth brushing pattern is determined to be identical.

A system according to an example embodiment of the disclosure may include an electronic device, a first external electronic device, and a second external electronic device.

According to an example embodiment of the disclosure, the electronic device may request first data related to an electronic medical record of a user of the electronic device from the second external electronic device, based on detecting a coupling between a first external electronic device and a first object related to a tooth brushing activity of the user. The second external electronic device may transmit the first data to the electronic device, based on the request. The electronic device may, based on receiving the first data from the second external electronic device, identify a position of a first tooth for providing a tooth brushing pattern, based on the first data, determine a first tooth brushing pattern corresponding to the identified position of the first tooth, and control the display to provide at least one first user guide, based on the determined first tooth brushing pattern.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used in connection with various embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various modifications, alternatives and/or variations of the various example embodiments may be made without departing from the true technical spirit and full technical scope of the disclosure, including the appended claims and their equivalents. It will also be understood that any of the embodiment(s) described herein may be used in conjunction with any other embodiment(s) described herein.

What is claimed is:

1. An electronic device comprising:
a wireless communication circuit;
a display;
at least one processor, comprising processing circuitry; and
a memory for storing instructions,
wherein at least one processor, individually and/or collectively, is configured to execute the instructions to cause the electronic device to:
identify first data related to an electronic medical record of a user, based on detecting a coupling between a first external electronic device and a first object related to a tooth brushing activity of the user;
determine a first tooth brushing pattern related to the tooth brushing activity of the user, based on the first data;
receive second data related to the tooth brushing activity of the user from the first external electronic device via the wireless communication circuit;
identify a second tooth brushing pattern indicated by the tooth brushing activity of the user, based on the second data;
determine a similarity between the first tooth brushing pattern and the second tooth brushing pattern; and
control the display to display guide information based on the first tooth brushing pattern, based on determining that the similarity is less than a specified threshold level.

2. The electronic device of claim 1, wherein at least one processor, individually and/or collectively, is configured to execute the instructions to cause the electronic device to:
based on obtaining the first data, control the display to display a first image indicating a user teeth condition, based on the first data.

3. The electronic device of claim 2, wherein at least one processor, individually and/or collectively, is configured to execute the instructions to cause the electronic device to:
determine a tooth corresponding to a position where the tooth brushing activity of the user is to begin, based on an input on the first image.

4. The electronic device of claim 1, wherein at least one processor, individually and/or collectively, is configured to execute the instructions to cause the electronic device to:
control the display to display a second image for guiding a tooth brushing order corresponding to the first tooth brushing pattern for portions of a tooth of the user.

5. The electronic device of claim 1, wherein at least one processor, individually and/or collectively, is configured to execute the instructions to cause the electronic device to:
control the display to display content identifying the user performing the tooth brushing activity, based on detecting the coupling between the first external electronic device and the first object.

6. The electronic device of claim 1, wherein the second data indicates the change of at least one of a roll value, a pitch value, a yaw value, and a pressure value that are detected by the first external electronic device coupled to the first object.

7. The electronic device of claim 2, wherein at least one processor, individually and/or collectively, is configured to execute the instructions to cause the electronic device to:
control the display to display a third image to which a specified effect generated based on the first image is applied,
wherein the specified effect comprises an effect of stepwisely changing a color of a tooth on which the tooth brushing activity of the user is being performed, based on a progress of the tooth brushing activity of the user.

8. The electronic device of claim 1, wherein the first external electronic device comprises a wearable device that, based on being decoupled from the first object, is worn on the user's body and configured to provide biometric information of the user to the electronic device.

9. A personalized tooth brushing guide method comprising:
identifying, by an electronic device, first data related to an electronic medical record of a user, based on detecting a coupling between a first external electronic device and a first object related to a tooth brushing activity of the user;
determining, by the electronic device, a first tooth brushing pattern related to the tooth brushing activity of the user, based on the first data;
receiving, by the electronic device, second data related to the tooth brushing activity of the user from the first external electronic device via a wireless communication circuit of the electronic device;
identifying, by the electronic device, a second tooth brushing pattern indicated by the tooth brushing activity of the user, based on the second data;
determining, by the electronic device, a similarity between the first tooth brushing pattern and the second tooth brushing pattern; and
displaying guide information based on the first tooth brushing pattern, based on determining that the similarity is less than a specified threshold level.

10. The method of claim 9, wherein the obtaining the first data comprises:
based on obtaining the first data, displaying a first image indicating a user teeth condition, based on the first data.

11. The method of claim 10, wherein the displaying the first image comprises:
determining a tooth corresponding to a position where the tooth brushing activity of the user is to begin, based on an input on the first image.

12. The method of claim 10, wherein the displaying the first image comprises:
displaying a third image to which a specified effect generated based on the first image is applied,
wherein the specified effect comprises an effect of stepwisely changing a color of a tooth on which the tooth brushing activity of the user is being performed, based on a progress of the tooth brushing activity of the user.

13. The method of claim 9, wherein the determining the first tooth brushing pattern comprises:
display a second image for guiding a tooth brushing order corresponding to the first tooth brushing pattern for portions of a tooth of the user.

14. The method of claim 9, further comprising:
displaying content identifying the user performing the tooth brushing activity, based on detecting the coupling between the first external electronic device and the first object

15. The method of claim 9, wherein the second data indicates the change of at least one of a roll value, a pitch value, a yaw value, and a pressure value that are detected by the first external electronic device coupled to the first object.

16. A non-transitory computer-readable storage medium on which a computer program is recorded,
wherein the computer program comprises instructions, which when executed by at least one processor, comprising processing circuitry, individually and/or collectively, of an electronic device, cause the electronic device to perform operations comprising:
identifying first data related to an electronic medical record of a user, based on detecting a coupling between a first external electronic device and a first object related to a tooth brushing activity of the user;
determining a first tooth brushing pattern related to the tooth brushing activity of the user, based on the first data;
receiving second data related to the tooth brushing activity of the user from the first external electronic device via a wireless communication circuit of the electronic device;
identifying a second tooth brushing pattern indicated by the tooth brushing activity of the user, based on the second data;
determining a similarity between the first tooth brushing pattern and the second tooth brushing pattern; and
controlling a display of the electronic device to display guide information based on the first tooth brushing pattern, based on determining that the similarity is less than a specified threshold level.

17. An electronic device comprising:
a wireless communication circuit;
a display;
at least one processor, comprising processing circuitry; and
a memory for storing instructions,
wherein at least one processor, individually and/or collectively, is configured to execute the instructions to cause the electronic device to:
identify a position of a first tooth to provide a tooth brushing pattern, based on first data about a dental treatment record;
determine a first tooth brushing pattern corresponding to the identified position of the first tooth; and
control the display to provide at least one first user guide, based on the determined first tooth brushing pattern.

18. The electronic device of claim 17, wherein the first tooth brushing pattern comprises at least one of a rolling comprising moving a toothbrush from top to bottom or from bottom to top of the first tooth, a pitching comprising moving the toothbrush from left to right or from right to left of the first tooth, and/or a pressing comprising pressing the toothbrush against the first tooth.

19. The electronic device of claim 17, wherein at least one processor, individually and/or collectively, is configured to execute the instructions to cause the electronic device to:
receive second data about a second tooth brushing pattern indicated by a tooth brushing activity of a user from a first external electronic device; and
control the display to provide the at least one first user guide, based on a comparison between the first tooth brushing pattern and the second tooth brushing pattern.

20. The electronic device of claim 17, wherein the first tooth comprises at least one or more teeth for which the first tooth brushing pattern is determined to be identical.
